(19) United States
(12) Patent Application Publication
(10) Pub. No.: US 2009/0048525 A1
(43) Pub. Date: Feb. 19, 2009

(54) Venous refill testing system and method

(75) Inventors: William Lee Rogers, Woodbury, MN (US); Marius Poliac, Reno, NV (US); Gary Warner Blanch, Burnsville, MN (US); Roger Charles Thede, Afton, MN (US); Daniel James Ince, Lakeville, MN (US); Andrew Paul Karels, Savage, MN (US); John Alexander Romans, North Oaks, MN (US); Michael Jerome Siers, Duluth, MN (US); Charles Fredric Steaderman, Burnsville, MN (US)

Correspondence Address: KINNEY & LANGE, P.A., THE KINNEY & LANGE BUILDING, 312 SOUTH THIRD STREET, MINNEAPOLIS, MN, 55415-1002, US

(73) Assignee: BioMedix, Inc., Vadnais Heights, MN (US)

(21) Appl. No.: 11/891,918

(22) Filed: Aug. 14, 2007

(51) Int. Cl. A61B 5/0295

(52) U.S. Cl. 600/504; 600/481

(57) ABSTRACT

A venous refill testing system includes a sensor for sensing patient blood volume at a testing location along a patient's leg during and after dorsiflexion of the patient's foot, and a controller unit having means for automatically determining a time of cessation of dorsiflexion of the patient's foot and for automatically evaluating whether a venous refill time following the time of cessation of dorsiflexion of the patient's foot is less than approximately twenty seconds. The sensor is operably connected to the controller unit, and the controller unit can be actuated to initiate sensing of patient blood volume with the sensor.

### BACKGROUND

The present invention relates to venous refill testing systems and methods.

In a patient's lower extremities, blood is pumped back upward and inward through a venous system. With a patient having generally healthy venous valves, movement of a patient's legs pumps blood through the venous system past a series of venous valves resulting in a pressure in the venous system of the lower leg that is nearly zero. As blood from the patient's arteries refills the leg veins, venous pressure is primarily the result of the hydrostatic pressure of blood present in the venous system to the nearest competent valve upstream. Therefore, in healthy patients, the refilling of the lower venous system following cessation of ambulation occurs relatively slowly. However, with venous insufficiency states in a patient's lower extremities, unhealthy valves allow venous blood to flow backward down the veins (called “venous reflux”), causing the lower venous system to refill relatively quickly. Generally speaking, venous insufficiency is present where venous valves are incompetent or otherwise unable to function in a healthy manner, and can be present in the patient's deep venous system and/or superficial venous system. Venous conditions can relate to other physiological conditions, for instance, chronic lower-extremity ulcers and varicosities.

Venous refill examination is a principal method for evaluation of peripheral superficial venous insufficiency, and has historically been performed utilizing a conventional photoplethysmography (PPG) probe that is driven by a DC coupled circuit. The examination is typically performed in a specialized vascular lab and administered by trained personnel. During examination, the patient is asked to perform exercises while the PPG probe provides patient venous system data to a standard strip chart recorder. The recorded strip chart information is mounted to a report form for manual visual evaluation by a medical specialist for diagnosis. However, this type of examination technique and reporting has led to technical difficulty and errors. For example, personnel administering the examination must be trained to ensure that recorded strip chart information is suitable for venous refill examination, meaning that examination generally can only occur at a specialized vascular lab where trained personnel are located rather than in primary care facilities (e.g., general practice clinics). But despite training, proper probe placement and signal strength evaluation are still heavily operator dependent and can introduce undesired test quality tolerances due to operator-induced variance. Moreover, when all potential venous insufficiency patients must be referred to a specialized vascular laboratory, primary diagnosis of this condition is burdensome and becomes cost prohibitive. Furthermore, reporting of the test results in this manner has proven to be inefficient and has delayed the reporting of test results to referring physicians and patients.

Numerous venous refill testing systems are known. For example, Newman et al., U.S. Pat. No. 5,050,613, entitled “Method and Apparatus for Vascular Testing”, discloses a system for performing bilateral venous refill testing (called venous reflux testing by Newman et al.). The disclosed system uses a PPG sensor to generate a waveform that represents raw PPG sensor measurements. Waveform data is sensed when a patient performs flexures and then continues after flexation has stopped. After test data has been collected, an operator manually places a first caliper 486 relative to the generated waveform at a location there a flexure region 484 ends. The system then performs an automatic re-scaling of the waveform display to offset hardware error. A second caliper 488 is then placed relative to the generated (and re-scaled) waveform, and a refill time calculated between the first and second calipers 486 and 488. The Newman et al. system presents a number of difficulties and concerns. The manual placement of the first caliper 486 remains heavily operator dependent, requiring a trained operator to visually assess the cessation of patient flexation, which can still undermine test quality and reproducibility due to operator-induced variance. Moreover, the automatic re-scaling by the Newman et al. system has lead to clinical unreliability of test results, because the rescaling disclosed by Newman et al. can distort amplitude on the waveform display, posing clinical reliability risks for subsequent diagnosis and interpretation.

### SUMMARY

A venous refill testing system includes a sensor for sensing patient blood volume at a testing location along a patient's leg during and after dorsiflexion of the patient's foot, and a controller unit having means for automatically determining a time of cessation of dorsiflexion of the patient's foot and for automatically evaluating whether a venous refill time following the time of cessation of dorsiflexion of the patient's foot is less than approximately twenty seconds. The sensor is operably connected to the controller unit, and the controller unit can be actuated to initiate sensing of patient blood volume with the sensor.

### BRIEF DESCRIPTION OF THE DRAWINGS

FIG. 1 is a block diagram of a venous refill testing system according to the present invention.

FIG. 2 is a display output of venous refill testing.

FIG. 3 is a flow chart illustrating a method of venous refill testing according to the present invention.

FIG. 4 is a flow chart illustrating a method for analyzing a venous refill period in conjunction with the method illustrated in FIG. 3.

FIG. 5 is a graph of exemplary venous refill testing data.

### DETAILED DESCRIPTION

In general, a venous refill testing system and method according to the present invention is used to measure a venous refill time of a superficial venous system of a patient's lower extremity (e.g., leg). The system and method of the present invention enables non-specialized operators, such as a primary care physician and his or her non-technician staff, to easily perform high quality venous refill diagnostic evaluations on a patient. Upon actuation, the system and method of the present invention prompts the operator to perform the venous refill diagnostic procedure while reducing or eliminating the number of operator-dependent decisions. An operator positions a sensor at a test location on a patient, then actuates the system to begin data collection. The patient performs a conventional venous refill testing dorisflexion procedure during data collection. The system automatically makes a determination as to whether an adequate and sufficient sensor signal is generated during data collection. The system also automatically identifies cessation of the dorsiflexion procedure to enable the system to analyze a venous refill time period against a clinical standard.

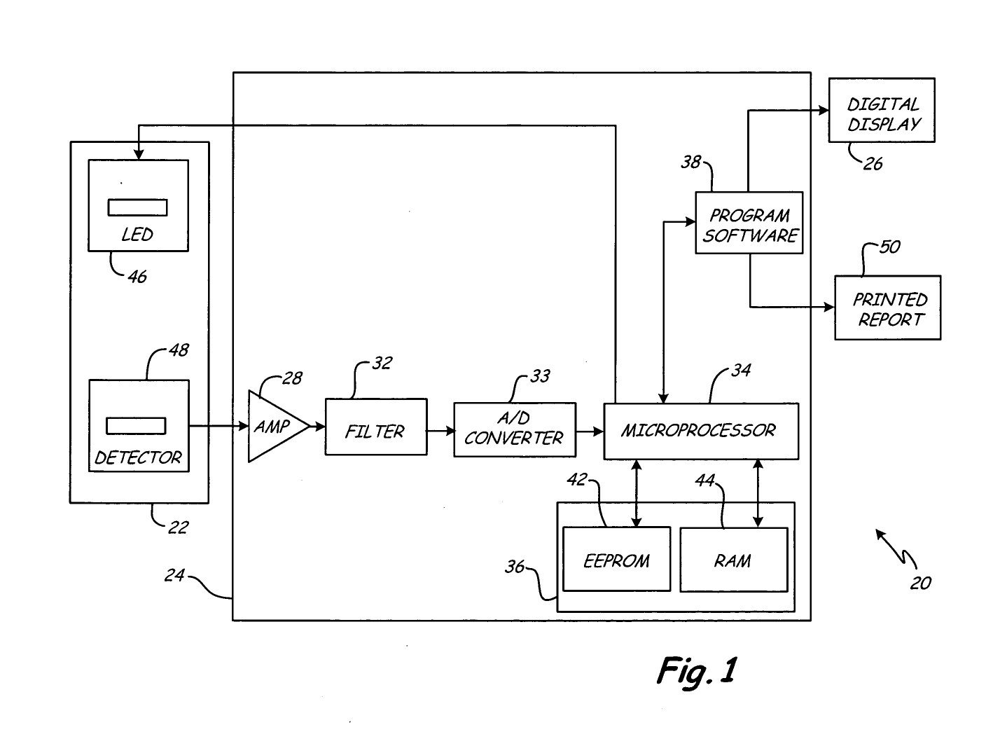

FIG. 1 is a block diagram of a venous refill testing system 20 that includes a direct current (DC) coupled photoplethysmography (PPG) sensor (or probe or transducer) 22, a controller unit 24, and a digital display 26. In the illustrated embodiment, the controller unit 24 includes a DC amplifier 28, a filter 32 (e.g., a low-pass filter), an analog-to-digital (A/D) converter 33, a microprocessor 34, memory 36, and program software 38. Components of the controller unit 24 can be implemented on a multi-layer printed circuit board (PCB). The memory 36 can include electrically erasable programmable read only memory (EEPROM) 42 and random access memory (RAM) 44. The PPG sensor 22 of the illustrated embodiment is of a conventional type and includes an infrared light-emitting diode (LED) 46 and a photo detector 48. Both the LED 46 and the photo detector 48 are housed in an opaque black plastic disk of the sensor 22, and are arranged to be flush with a common exterior surface of the disk. The LED 46 can be powered on whenever the controller unit 24 is actuated, and separate control circuitry is not necessary but could be provided in further embodiments. It should be noted that the configuration of components of the system 20 can vary from those shown in FIG. 1. For instance, additional components not shown can be included. Moreover, components such as the amplifier 28 can be incorporated into the PPG sensor 22 rather than the controller unit 24 in alternative embodiments.

The program software 38 can contain a patient database as well as operational software that drives operation of the system 20. The program software 38 can also perform signal filtering, including additional low-pass filtering beyond that provided by the filter 32. Patient demographic, history and physical examination can be completed and stored in the patient database prior to or after performing a venous refill test. The program software 38 can contain instructions for display on the digital display 26 to prompt the operator as desired, for example to explain proper application of the PPG sensor 22 to a suitable test location.

During operation, the LED 46 emits light in the infrared spectrum into subcutaneous tissue of the patient at a test location. In turn, light emitted from the LED 46 reflects back to the photo detector 48 as a function of blood volume at the test location, which correlates to density of red blood cells in cutaneous capillaries at the test location. The photo detector 48 sends a signal to the DC amplifier 28 of the controller unit 24, which can perform normalization in addition to amplification. The signal is also sent through the low pass filter 32, the A/D converter 33, and eventually the amplified signal is processed by the program software 38 for display as a waveform on the digital display 26 or a printed report 50, and/or stored to the patient database component of the program software 38. Signal gain can be adjusted.

The signal from the photo detector 48 of the PPG sensor 22 represents sensed patient data, and a change in the sensed patient data over time indicates changes in blood volume (i.e., blood content) in the skin at the test location. The skin blood content, that is, cutaneous blood content, is correlated with venous pressures, which develop as a function of arterial inflow to the test location and venous outflow and/or venous reflux from the test location. As explained in greater detail below, a decrease in cutaneous blood content at the test location causes the signal waveform to descend on the display, and an increase in blood content at the test location will elevate the signal waveform.

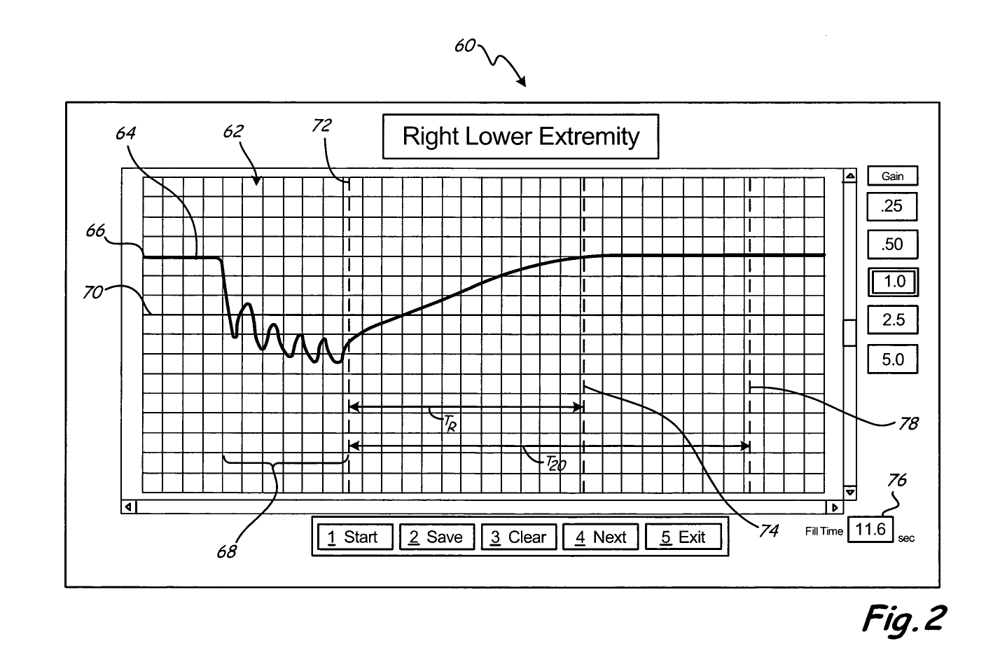

FIG. 2 shows an exemplary venous refill testing display output 60 for a patient's right lower extremity. The display output 60 can be indicated on the digital display 26, the printed report 50, or elsewhere, as desired. The display output 60 includes a grid 62, which is arranged to indicate time along a horizontal axis and a PPG sensor signal (e.g., blood volume) along a vertical axis. A waveform 64 generated as a function of the signal from the PPG sensor 22 is plotted on the grid 62. When the controller unit is actuated, the patient data is sensed and the waveform 64 can be generated beginning from an origin 66 along the vertical axis that corresponds to the first received data point of the signal from the PPG sensor 22. While PPG sensing continues, the patient performs a dorsiflexion procedure (explained further below), which is shown in a dorsiflexion region 68 of the waveform 64. The dorsiflexion region 68 generally exhibits oscillations in the waveform 64 and has an overall downward slope that corresponds to a period when exercises are performed by the patient during the test procedure, which tend to pump blood away from the test location. The oscillations in the dorsiflexion region 68 result from blood flow, because some refill generally occurs between successive exercise movements (i.e., dorsiflexion maneuvers) that each pump blood away. In the illustrated embodiment, the dorsiflexion procedure has caused the waveform 64 (and the signal upon which it is based) to drop below a threshold 70, which establishes a minimally sufficient amount of blood that must be pumped out of the patient's extremity in order to reliably assess a subsequent venous refill time period against clinically accepted standards.

Upon cessation of the dorsiflexion procedure, a venous refill period begins at a fill position 72. In the venous refill period, the waveform 64 generally exhibits an upward slope as blood refills the test location. An end of the venous refill period 74 occurs when the waveform 64 reaches the origin 66 in the vertical direction of the grid 62. A refill time (or fill time) T_{R} is established as the time (measured along the horizontal axis of the grid 62) between the fill position 72 and the end of the venous refill period 74. In the illustrated embodiment, the display output 60 also shows the fill time T_{R} (where T_{R}=11.6 seconds in the illustrated example) in a fill time display 76.

Regions of the grid 62 can be shaded to highlight selected information, and render the display output 60 more easily and rapidly understandable. Venous refill times are typically compared to a twenty second time period T₂₀, which can be lightly shaded on the grid 62 beginning at the fill position 72 and terminating at end point indication 78. The refill (or fill) time T_{R} can be more darkly shaded on the grid 62 between the fill position 72 and the end of the venous refill period 74. In this way, the refill time T_{R} and the twenty second time period T₂₀ can be shown relative to the waveform 64 to facilitate review and patient diagnosis. It should be noted that a “healthy” venous refill time is generally greater than twenty seconds, and may be greater than a testing window. Thus, where the refill time T_{R} may not be specifically calculated or shaded on the grid 62 for some test procedures.

Furthermore, the controller unit 24 calculates and establishes the values reported on the display output 60 by default. However, an operator can manually override values calculated and established by the controller unit 24 as desired for particular test procedures.

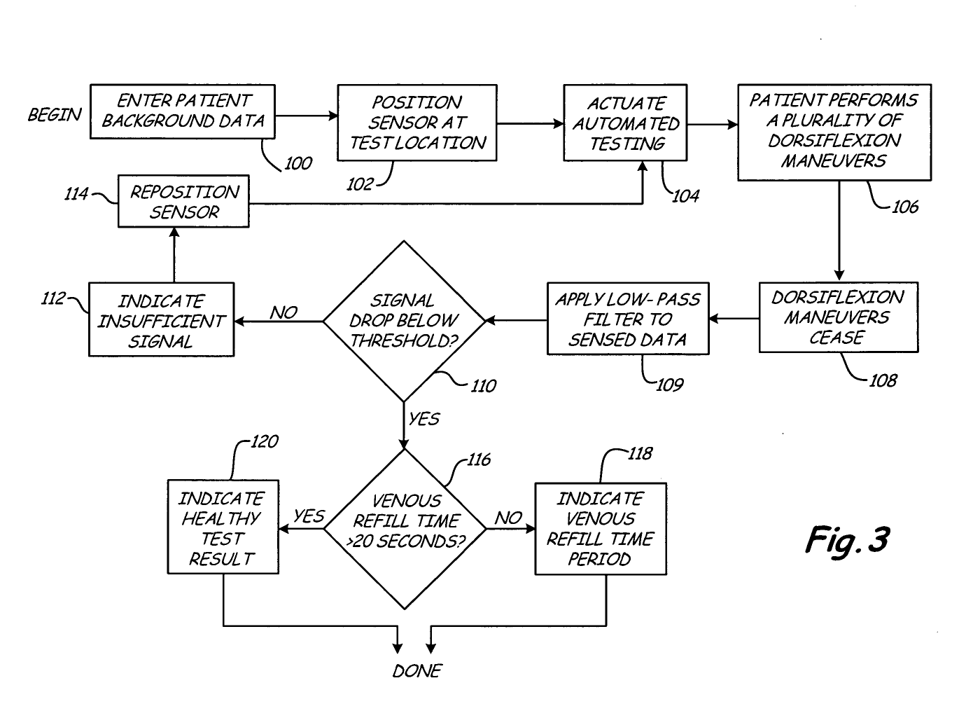

FIG. 3 is a flow chart illustrating one embodiment of a method of venous refill testing. Initially, an operator can enter relevant patient background data into the system 20 (step 100). Such patient background information could be entered at a later time in alternative embodiments. Then the operator places the PPG sensor 22 on the patient at a test location on a selected leg (step 102). The test location can be located approximately three finger-breadths (i.e., the width of three adjacent fingers) above a medial malleolus of the patient's leg and posterior of a midline of the patient's leg. At this point, the operator can instruct the patient to sit in a relaxed position with feet flat on the floor and, once testing beings, to perform a dorsiflexion procedure that includes five dorsiflexion maneuvers where the patient flexes his or her ankle to lift his or her foot from the floor. The program software 38 can prompt the operator regarding probe placement and patient instructions.

Once the PPG sensor 22 is positioned, and the patient is prepared for testing to begin, the operator then actuates the controller unit 24 to begin an automated testing procedure (step 104). The system 20 then begins sensing patient data for the venous refill test. While patient data is being sensed, the patient performs a plurality of dorsiflexion maneuvers (step 106), which cease while the system 20 continues to sense patient data (step 108). The system can continue sensing patient data with the PPG sensor 22 for the duration of a selected testing window, for example, a testing window lasting thirty seconds.

Filtering (e.g., low-pass hardware filtering provided by the filter 32) is applied to sensed data (step 109). Next, the controller unit 24 of the system 20 makes a determination as to signal sufficiency (or quality) during testing. Signal sufficiency is analyzed by determining whether the signal, after being amplified and filtered, drops below a threshold during the dorsiflexion procedure (step 110). The threshold corresponds to a suitable drop in blood volume at the test location to enable venous refill time to be appropriately measured in the time period that follows cessation of the dorsiflexion procedure. A suitable drop in blood volume will generally correspond to a refill time of greater than twenty seconds for a healthy patient. A sufficient drop can be measured as a particular distance on a graph that plots the signal as a waveform (e.g., a 15 mm vertical drop on a 40 mm signal waveform graph at a gain setting of 1.0). The upper point for determining the sufficiency of the drop can be the first data point of the sensed signal following amplification and filtering. Inadequate or insufficient drops in the signal can result, for example, from the PPG sensor 22 being positioned directly over a blood vessel where blood volume fluctuates but does not decrease over time during dorsiflexion maneuvers, making the testing location poor or unsuitable. Inadequate or insufficient drops in the signal can also result from insufficient performance of dorisflexion maneuvers by the patient.

If the PPG sensor 22 generates an inadequate signal (i.e., one that does not drop below the threshold), the display 36 of the system 20 will indicate to the operator that the signal was insufficient (step 112), and can prompt the operator to reposition the PPG sensor 22 and have the patient perform a new dorsiflexion procedure for a new test procedure (step 114). The operator can then actuate the system 20 again and repeat the venous refill testing procedure from step 104 onward. When an adequate signal is detected by the controller unit 24, the data collection will continue through the close of the testing window at which time venous refill time will be evaluated.

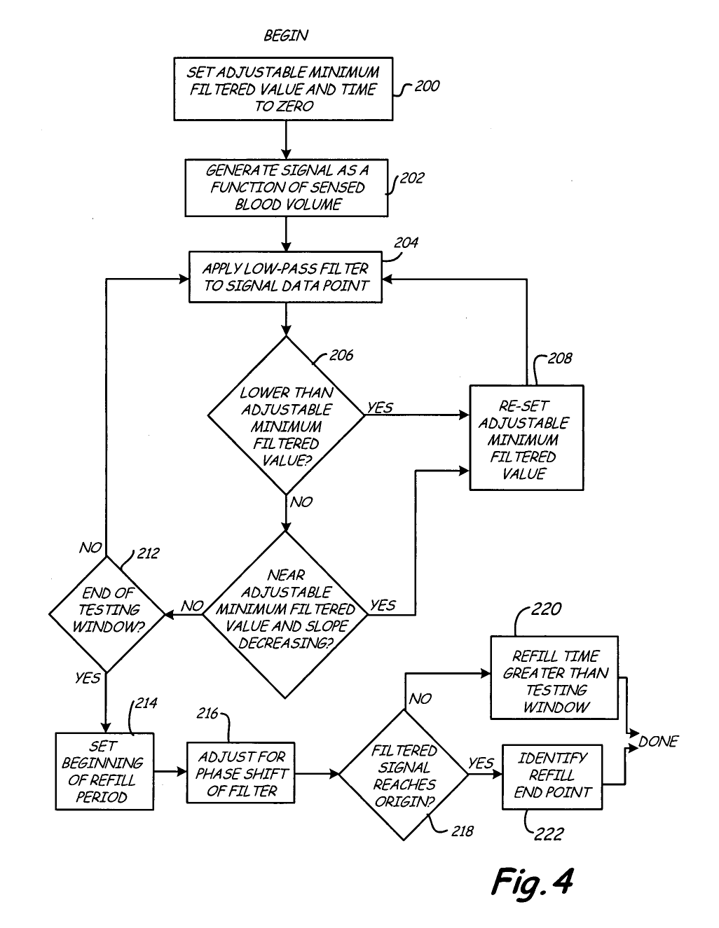

Upon the close of the testing window, the system 20 determines if venous refill time was greater than twenty seconds (step 116). The particular method for calculating venous refill time is explained further below with respect to FIG. 4. In published literature, a comparison of venous refill time to twenty second window is a generally accepted metric for distinguishing healthy venous refill times from potentially physiologically problematic venous refill times. Once the determination regarding the venous refill time at step 116 is made, the system 20 indicates either a specific venous refill time if the venous refill time is less than or equal to twenty seconds (step 118), or a “healthy” or generally non-problematic result if the venous refill time is greater than twenty seconds (step 120). The indications at steps 118 and 120 can be made via the digital display 26, the printed report 50, or other suitable manner in further embodiments. Additional detail as to how information can be indicated by the system 20 is discussed below.

In addition, the system 20 will document the resultant signal waveform from the test procedure. Subsequently, the operator will be prompted on the digital display 26 to save or repeat the venous refill testing procedure. The operator will also have the option to manually override calculations by the controller unit 24. The saved patient testing data is stored in the patient database component of the program software 38 and is available for printing on the printed report 50 or other transmittal options, such as transmission over the Internet to a server in the manner similar to that disclosed in commonly-assigned U.S. patent application Ser. No. 10/227,770, entitled SYSTEM AND METHOD FOR TESTING FOR CARDIOVASCULAR DISEASE, which is hereby incorporated by reference in its entirety.

FIG. 4 is a flow chart illustrating a method for analyzing a venous refill period in conjunction with the method described above with respect to FIG. 3. As a venous refill test is performed using the system 20, sensed venous data is monitored to detect a starting point of the venous refill period, that is, the point in time along the signal waveform when the patient ceases dorsiflexion and blood begins to flow back into the patient's extremity. The following algorithm allows detection of the start of the venous refill period and the length of the refill time period.

Initially, at the beginning of a test procedure, an adjustable minimum filtered value, and a time associated with that value, are set to zero (step 200). The adjustable minimum filtered value acts as a variable that allows data tracking and comparison through the test procedure. While the test procedure is underway, a signal is generated by the PPG sensor 22 as a function of sensed blood volume at the test location (step 202). Low-pass software filtering (e.g., low-pass software filtering provided by the program software 38) is applied to each data point in the signal (step 204), which produces a current filtered value for each data point in the signal.

For each data point in the signal, two analyses can be made. First, the current filtered value is compared to the adjustable minimum filtered value (step 206), and if the current filtered value of a given signal data point is less than the adjustable minimum filtered value then the adjustable minimum filtered value is re-set to the current filtered value (step 208).

If the current filtered value of the given signal data point is greater than or equal to the adjustable minimum filtered value at step 206, then a second analysis is performed (step 210). Generally, this second analysis at step 210 detects whether the dorsiflexion procedure is still underway, even if the dorsiflexion maneuver underway does not result in a current filtered value that is an absolute minimum or otherwise lower than the adjustable minimum filtered value. Such situations can arise because of the manner in which the patient performs the dorsiflexion procedure, for example, when a patient performs too many dorsiflexion maneuvers (e.g., performing many more than instructed) or pauses excessively between dorsiflexion maneuvers. The analysis at step 210 involves determining if the current filtered value is near the adjustable minimum filtered value and if a slope of a waveform representing current filtered values over time is decreasing. In one embodiment, the current filtered value can be considered near the adjustable minimum filtered value if the current filtered value is within 10% of the adjustable minimum filtered value. The slope of the waveform representing current filtered values over time can be analyzed mathematically by taking derivatives (e.g., a decreasing slope being indicated by a second derivative with a negative value). In an alternative embodiment, slope of the waveform can be compared to a selected slope threshold, such that the comparison is satisfied only when the slope is decreasing significantly, that is, beyond the selected threshold. If the current filtered value is near the adjustable minimum filtered value and if the slope of the waveform representing current filtered values over time is decreasing, then the adjustable minimum filtered value is re-set to the current filtered value (step 208).

If the current filtered value is not near the adjustable minimum filtered value or if the slope of the waveform representing current filtered values over time is not decreasing, then data collection proceeds to the: end of a testing window in which data samples are collected. The testing window can be a selected time for the generation of the signal at step 202 to continue, for instance, a time period of thirty seconds or another desired time period. A determination is made by the controller unit 24 as to when the end of the testing window is reached (step 212). If the testing window is not completed, then the next data point is filtered and analyzed in the same manner described above (go to step 204). If the testing window is complete, then data collection can cease, and an analysis of the venous refill period begun. Analysis of the venous refill period is performed after data collection (i.e., sensing with the PPG sensor 22) is finished.

In order to analyze the venous refill period, a point where the dorsiflexion procedure ends and the venous refill period begins is determined. The beginning of the venous refill period is set at a time when the last received “raw” signal value was greater than the adjustable minimum filtered value (step 214). For purposes of this analysis, the raw signal value refers to a value of the signal prior to low-pass software filtering by the program software 38, though some noise reduction by the filter 32 can have been performed on the raw signal. Next, the time set in step 214 as the beginning of the venous refill period can be adjusted for a phase shift of the filter 32 (step 216). Then an analysis related to the end of the refill period is performed. A determination is made as whether the raw signal value reaches the origin (step 218). The origin, for purposes of the determination at step 218, can be a first data point of the signal generated by the PPG sensor 22 (i.e., the first raw signal value) after the test procedure begins, collected before the patient performs the dorsiflexion procedure. In this way, the origin provides a reference as to blood volume prior to the dorsiflexion procedure, which allows an end point of the venous refill period to be established, as appropriate, at a time when the refilling of blood at the test location has compensated for blood pumped away during the dorsiflexion procedure (step 220). If the raw signal value does not reach the origin within twenty seconds of the beginning of the refill period, particularly if the raw signal value does not reach the origin within the testing window, then the end point and duration of the venous refill period may not be specifically determined. So long as the venous refill period is established to be longer than twenty seconds, a generally healthy test outcome is established (step 222), generally making calculation of the exact venous refill time unnecessary. It should be noted that the raw signal value can be determined to reach the origin when the raw signal value crosses the origin or when it enters a selected band surrounding the origin.

The present invention allows reliable venous refill testing that is relatively easy to administer, and which reduces a risk of operator-induced error in the testing process. For instance, operator-induced error resulting from reliance upon manual visual analysis of signal waveforms to identify cessation of a dorsiflexion procedure and the duration of a venous refill period is reduced or eliminated. Venous refill testing according to the present invention can be utilized to screen patients to detect the presence of disease, while the location and treatment of specific health problems detected can be addressed later by appropriate medical specialists.

### EXAMPLE

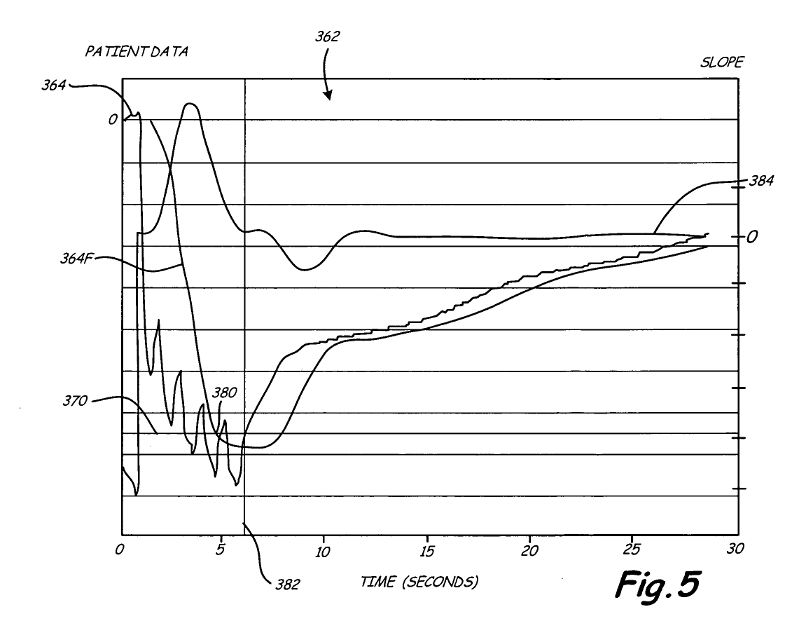

An example venous refill test procedure was performed using the system and method of the present invention as disclosed above. A PPG sensor 22 was positioned at a test location three finger-breadths above a medial malleolus and posterior of a midline of a leg of a generally healthy test subject, and a controller unit 24 was actuated to begin data collection. FIG. 5 is a graph 362 of resultant venous refill testing data from the test procedure. The graph 362 illustrates some information that would not be visually displayed by a test system 20 in a typical embodiment, but shows such extra information for illustrative purposes of the present example. In FIG. 5, a waveform 364 plotted on the graph 362 represents “raw” signal data obtained from the PPG sensor 22 (upon which some noise reduction can have been performed, but prior to low-pass software filtering). A filtered waveform 364F is also plotted on the graph 362 that represents signal data corresponding to the waveform 364 after being filtered with low-pass software filtering provided by the program software 38. The test subject performed five dorsiflexion maneuvers during an approximately thirty second test window. During the dorsiflexion procedure, the filtered waveform 364F dropped below a threshold 370 at a point 380, which indicated that blood had been sufficiently pumped from the test location for venous refill testing.

A fill position 382 was established in the example test procedure at the beginning of the venous refill period following cessation of the dorsiflexion procedure. This was accomplished by tracking an adjustable minimum filtered value (not shown) and a slope of the filtered waveform 364F. The slope of the filtered waveform 364F (i.e., its first derivative) is plotted on the same graph as a slope waveform 384 (the vertical axis for the slope is shown at the right of the graph 362 and the vertical axis for the filtered and raw waveforms 364F and 364 are shown at the left of the graph 362 in FIG. 5). In the example, the adjustable minimum filtered value was identical to the filtered waveform 364F up to a point just before the horizontal axis (time) location when the fill period 382 was established. Then, at the time corresponding to the fill period 382, when venous refill at the test location began, the filtered waveform 364F increased above the minimum filtered value, and continued to increase through the end of the test window. For that reason, the adjustable minimum filtered value remained set at the absolute minimum filtered value, and there was no need to re-set the adjustable minimum filtered value for other reasons (i.e., the filtered waveform 364F was never greater than but near the adjustable minimum filtered value with a decreasing slope). The fill position 382 was established at a time when the waveform 364 rose above the adjustable minimum filtered value (which was also the absolute minimum filtered value in the example).

The waveform 364 did not rise to reach an origin (indicated as zero at the left side “patient data” axis of the graph 362) within twenty seconds of time at which the fill period 282 was established. In other words, the venous refill time was established to be greater than twenty seconds. The specific refill time was not calculated in the example, but was determined to be within accepted normal range after being established to have a duration greater than twenty seconds. Thus, in the present example, the venous refill test did not indicate an abnormal or problematic venous refill period, and a generally healthy result was indicated.

Although the present invention has been described with reference to preferred embodiments, workers skilled in the art will recognize that changes may be made in form and detail without departing from the spirit and scope of the invention.

1. A venous refill testing system comprising:
a sensor for sensing patient blood volume at a testing location along a patient's leg during and after dorsiflexion of the patient's foot; and
a controller unit having means for automatically determining a time of cessation of dorsiflexion of the patient's foot and for automatically evaluating whether a venous refill time following the time of cessation of dorsiflexion of the patient's foot is less than approximately twenty seconds, wherein the sensor is operably connected to the controller unit, and wherein the controller unit can be actuated to initiate sensing of patient blood volume with the sensor.

2. (canceled)

3. The system of claim 1, the controller unit further comprising:
a low pass filter for filtering the signal to produce a filtered signal.

4. (canceled)

5. The system of claim 4, the controller unit further comprising:
means for determining a slope of a waveform that corresponds to the filtered blood signal over time, and for adjusting the adjustable minimum filtered value as a function of the filtered signal, nearness of the filtered signal to the adjustable minimum filtered signal at a given instant, and the slope of the waveform.

6. The system of claim 1, the controller unit further comprising:
means for determining whether dorsiflexion of the patient's foot has sufficiently reduced blood volume at the testing location for venous refill testing.

7. The system of claim 6, wherein dorsiflexion of the patient's foot has sufficiently reduced blood volume at the test location for venous refill testing when any data point within the sensed patient data corresponds to a blood volume value less than a blood volume threshold value.

8. The system of claim 7 and further comprising:
a display operably connected to the controller unit for displaying a waveform as a function of the sensed patient data, wherein the blood volume threshold value is established at a fixed vertical distance below a first data point of the sensed patient data.

9. (canceled)

10. The system of claim 1, wherein the sensor comprises a photoplethysmography transducer.

11. A method for venous testing, the method comprising:
initiating testing with an automatic testing apparatus;
sensing patient data at a test location along a patient's leg using the automatic testing apparatus;
instructing a patient to perform dorsiflexion of the patient's foot while the automatic testing apparatus is sensing patient data, wherein dorsiflexion of the patient's foot causes a decrease in blood volume in the patient's leg;
determining as a function of the sensed patient data whether the decrease in blood volume in the patient's leg has passed a threshold; and
if the blood volume in the patient's leg has passed the threshold, tracking a venous refill time period that begins upon cessation of dorsiflexion of the patient's foot.

12. The method of claim 11 and further comprising:
determining whether the venous refill time period is less than twenty seconds, wherein an end of the venous refill time period is established where blood volume at the test location rises to at least an original level sensed by the automatic testing apparatus before dorsiflexion of the patient's foot.

13. The method of claim 12 and further comprising:
if the venous refill time period is less than twenty seconds, calculating the venous refill time in seconds.

14. The method of claim 11 and further comprising:
displaying a waveform as a function of sensed patient data; and
indicating the venous refill time period relative to the waveform.

15. The method of claim 11 and further comprising:
displaying a waveform as a function of sensed patient data; and
indicating a twenty second time period relative to the waveform, wherein a beginning of the twenty second time period is aligned to the beginning of the venous refill time period.

16. The method of claim 11, wherein steps for establishing cessation of dorsiflexion of the patient's foot comprise:
setting an adjustable minimum filtered blood volume value to zero when testing with an automatic testing apparatus is initiated;
sampling a plurality of patient data points over time with a sensor of the automatic testing apparatus;
applying a low pass filter to a sensor signal from the sensor to produce a current filtered blood volume value as each patient data point is sampled;
determining a slope of a waveform that corresponds to the current filtered blood volume values over time;
if the current filtered blood volume value is less than the minimum filtered blood volume value for each patient data point sampled, adjusting the minimum filtered blood volume value to the current filtered blood volume value;
if the current filtered blood volume value is near of the minimum filtered blood volume value and the slope of the waveform that corresponds to the current filtered blood volume values over time is decreasing significantly, adjusting the minimum filtered blood volume value to the current filtered blood volume value; and
establishing a time value corresponding to cessation of dorsiflexion of the patient's foot as a last patient data point where the current filtered blood volume value is greater than the minimum filtered blood volume value.

17. A method of venous refill testing, the method comprising:
sensing a parameter that corresponds to blood volume;
generating a signal as a function of the parameter;
determining whether the parameter has dropped below a threshold based upon analysis of the signal;
determining a time when dorsiflexion has ceased based upon analysis of the signal;
analyzing a venous refill time that begins at the time when dorsiflexion has ceased; and
providing an indication of results of the step of analyzing the venous refill time.

18. The method of claim 17 and further comprising:
determining whether the venous refill time is less than twenty seconds, wherein an end of the venous refill time is established where the signal rises to at least an original level before dorsiflexion.

19. The method of claim 18 and further comprising:
if the venous refill time is less than twenty seconds, calculating a venous refill time value in seconds.

20. (canceled)

21. The method of claim 17, wherein the dorsiflexion procedure comprises a plurality of dorsiflexion maneuvers.

22. A venous test system comprising:
a sensor for sensing patient data at a test location along a patient's leg during and after dorsiflexion of the patient's foot and for producing a signal indicative of patient blood volume; and
a processor unit for processing the sensed patient data, wherein the processor unit is configured to evaluate the sensed patient data to determine whether dorsiflexion of the patient's foot has sufficiently reduced blood volume at the test location for venous refill testing that includes evaluation of venous refill time following cessation of dorsiflexion of the patient's foot.

23. (canceled)

24. The system of claim 22, the processor unit further comprising:
means for automatically evaluating whether the venous refill time following cessation of dorsiflexion of the patient's foot is less than twenty seconds.

25. The system of claim 22, the processor unit further comprising:
a low pass filter for filtering the sensed patient data to produce a filtered signal.

26. The system of claim 25, the processor unit further comprising:
means for adjusting an adjustable minimum filtered value as a function of the filtered signal.

27. The system of claim 26, the processor unit further comprising:
means for determining a slope of a waveform that corresponds to the filtered blood signal over time, and for adjusting the adjustable minimum filtered value as a function of the filtered signal, nearness of the filtered signal to the adjustable minimum filtered signal at a given instant, and the slope of the waveform.

28. The system of claim 25, wherein dorsiflexion of the patient's foot has sufficiently reduced blood volume at the test location for venous refill testing when any data point within the sensed patient data corresponds to a blood volume value less than a blood volume threshold value.

29. The system of claim 28 and further comprising:
a display for displaying a waveform as a function of the sensed patient data, wherein the blood volume threshold value is established at a fixed vertical distance below a first normalized data point of the sensed patient data.

30. The system of claim 22 and further comprising:
a display for displaying a waveform as a function of the sensed patient data.

31. (canceled)

32. The system of claim 22, wherein the sensor comprises a photoplethysmography transducer.

33. The system of claim 1, wherein dorsiflexion of the patient's foot comprises a plurality of flexation events.

34. The system of claim 3, the controller unit further comprising:
means for adjusting an adjustable minimum filtered value as a function of the filtered signal.

35. The system of claim 1 and further comprising:
a display operably connected to the controller unit for displaying a waveform as a function of the signal.

36. The method of claim 17 and further comprising:
displaying a waveform as a function of the signal.

37. The system of claim 22, wherein dorsiflexion of the patient's foot comprises a plurality of flexation events.
